(19) United States
(12) Patent Application Publication
(10) Pub. No.: US 2025/0312495 A1
(43) Pub. Date: Oct. 9, 2025

(54) ORGANOMETALLIC GOLD(III) COMPLEXES FOR RADIOLABELING BIOMOLECULES FOR APPLICATIONS IN POSITRON EMISSION TOMOGRAPHY (PET) MOLECULAR IMAGING

(71) Applicant: The Regents of the University of California, Oakland, CA (US)

(72) Inventors: Jennifer M. Murphy, Los Angeles, CA (US); James Wells McDaniel, Los Angeles, CA (US); Alexander Spokoyny, Woodland Hills, CA (US); Julia Stauber, San Diego, CA (US); Evan Ambrose Doud, Los Angeles, CA (US)

(73) Assignee: The Regents of the University of California, Oakland, CA (US)

(21) Appl. No.: 18/867,577

(22) PCT Filed: Jun. 8, 2023
(86) PCT No.: PCT/US23/68142

§ 371 Date: Nov. 20, 2024

Related U.S. Application Data
(60) Provisional application No. 63/350,543, filed on Jun. 9, 2022.

(51) Int. Cl. A61K 51/04; C07F 1/00

(52) U.S. Cl. CPC A61K 51/0478; C07F 1/00

(57) ABSTRACT

The ¹⁸F-labeling of unprotected peptides and sugars via thioarylation using a Au(III)-[^{˜F}]fluoroaryl complex is reported. The chemoselective method generates ¹⁸F-labeled S-aryl bioconjugates in an aqueous environment in 15 min with high radiochemical yields and displays excellent functional group tolerance. This approach utilizes an air and moisture stable, robust organometallic Au(III) complex and highlights the versatility of designer organometallic reagents as efficient agents for rapid radiolabeling.

### CROSS REFERENCE TO RELATED APPLICATIONS

This application claims the benefit under 35 U.S.C. Section 119(e) of co-pending and commonly-assigned U.S. Provisional Patent Application No. 63/350,543, filed Jun. 9, 2022, entitled “ORGANOMETALLIC GOLD (III) COMPLEXES FOR RADIOLABELING BIOMOLECULES FOR APPLICATIONS IN POSITRON EMISSION TOMOGRAPHY (PET) MOLECULAR IMAGING”, which application is incorporated by reference herein.

### STATEMENT REGARDING FEDERALLY SPONSORED RESEARCH AND DEVELOPMENT

This invention was made with government support under Grant Number GM124746, awarded by the National Institutes of Health. The government has certain rights in the invention.

### TECHNICAL FIELD

The invention relates to agents for imaging targets such as molecules, cells and organs, and compositions and methods for making and using such agents.

### BACKGROUND OF THE INVENTION

The most common ¹⁸F-labeling method for biomolecules to date, utilizes ¹⁸F-SFB, a radiolabeled prosthetic group that reacts with the c-amino group of surface-exposed lysine residues (Liu et al., 2011, Mol. Imaging 10:168; Cai et al., 2007, J. Nucl. Med. 48:304; Olafsen et al., 2012, Tumor Biol. 33:669). In addition, site-specific conjugation using 4-¹⁸F-fluorobenzaldehyde (18-FBA) has also been demonstrated (Cheng et al., 2008, J. Nucl. Med. 49:804). While ¹⁸F-SFB has been successfully used to generated ^{I}T-labeled proteins and peptides, labeling with ¹⁸F-SFB is far from ideal; in addition to its unselective conjugation, its 3-step synthesis and subsequent protein conjugation results in very poor decay-corrected radiochemical yields of 1.4-2.5%.

There is a need in the art for materials and methods useful for making and/or using new ¹⁸F-labeled compounds. The present invention addresses this unmet need.

### SUMMARY OF THE INVENTION

The present invention provides materials and methods useful for the radiolabeling of biomolecules such as peptides and sugars. Typical methods of the invention utilize an air and moisture stable, robust organometallic Au(III) complex selected for its ability to effect this labelling, with the working examples of the invention highlighting the versatility of the organometallic reagents disclosed herein as efficient agents for rapid radiolabeling. As discussed below, using an Au(III)-[¹⁸F]fluoroaryl complex, the chemoselective methods disclosed herein can generate ¹⁸F-labeled S-aryl bioconjugates in an aqueous environment in 15 min with high radiochemical yields. In addition to the observed rapid radiolabeling reactions, the methods and associated materials disclosed herein further display excellent functional group tolerance.

The invention disclosed herein has a number of embodiments. Embodiments of the invention include, for example, compositions of matter including an oxidative addition reagent as disclosed herein. Typically, the oxidative addition reagent includes Au(III), a ligand bound to Au(III), the ligand comprising a phosphine, polydentate and/or monodentate ligand; and an aryl or heterocycle ring coupled to a ¹⁸F or ¹¹C moiety. When such oxidative addition reagents are combined with a biomolecule comprising a sulfur or selenium atom (e.g., in an aqueous solution); the oxidative addition reagent reacts with the biomolecule so as to covalently link the ¹⁸F or the ¹¹C moiety to the biomolecule. In certain embodiments of the invention, the composition further comprises a polypeptide selected to comprise a sulfur atom (optionally one coupled to an ¹⁸F moiety); and/or a polysaccharide selected to comprise a sulfur atom (optionally one coupled to an ¹⁸F moiety). In certain embodiments of the invention, the composition comprises an aqueous media; an aqueous buffering agent; and/or an alcohol. In one embodiment of the invention, the oxidative addition reagent comprises a compound having a general formula as shown in FIG. 3.

Embodiments of the invention also include methods of making the compositions disclosed herein. Typically, these methods comprise combining together to make an oxidative addition reagent, Au(III), a ligand bound to Au(III), the ligand comprising a phosphine, polydentate and/or monodentate ligand; and an aryl or heterocycle ring coupled to a ¹⁸F or ¹¹C moiety. Optionally, the compositions further comprise a polypeptide selected to comprise a sulfur atom; and/or a polysaccharide selected to comprise a sulfur atom. In certain embodiments of the invention, the method comprises including in the combination at least one of an aqueous media, an aqueous buffering agent (e.g., a PBS or TRIS buffering agent), and/or an alcohol. In illustrative working embodiments of the invention, the composition comprises a TRIS buffer/methanol solvent system.

Other embodiments of the invention include methods of coupling ¹⁸F or ¹¹C to a sulfur or selenium atom. Typically, these methods comprise combining together an oxidative addition reagent comprising ¹⁸F or ¹¹C; a solvent (e.g an aqueous media); a polypeptide selected to comprise a sulfur (or selenium) atom; and/or a polysaccharide selected to comprise a sulfur (or selenium) atom. In such methods, the combination undergoes an oxidative addition reaction such that ¹⁸F or ¹¹C is coupled to a sulfur or selenium atom present on the polypeptide selected to comprise a sulfur or selenium atom; and/or the polysaccharide selected to comprise a sulfur or selenium atom (e.g., so as to form an ¹⁸F or ¹¹C imaging agent useful in positron emission tomography). In certain embodiments of these methods, the oxidative addition reaction occurs at a temperature of 45° C. or below. In certain embodiments of these methods, the oxidative addition reaction is allowed to proceed for less than 60, 30 or 15 minutes. Optionally, the methods generate [¹⁸F] or [¹¹C] coupled polypeptides or polysaccharides in an at 80% or 90% radiochemical yield (RCY).

Other embodiments of the invention include methods of utilizing the [¹⁸F] or [¹¹C] coupled polypeptides or polysaccharides made by the methods disclosed herein in a positron emission tomography (PET) process. For example, embodiments of the invention include methods for imaging a biological target by PET scanning, the method comprising combining a ¹⁸F or [¹¹C] labeled imaging agent generated by a method disclosed herein with the biological target, and using the imaging agent to image the target.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Schematics showing (a) ¹¹C- and ¹⁸F-labeling of unprotected peptides via Pd-mediated S-arylation; and (b) an embodiment of the invention showing ¹⁸F-labeling of unprotected peptides, sugars and β-cyclodextrin via Au-mediated S-arylation.

FIG. 2. Schematic showing ¹⁸F-Labeling of molecules via AuIII-mediated S-arylation. Reaction conditions: substrate (5 mg), [¹⁸F]1 (0.5-2.0 mCi), Tris buffer pH 8.0 (750 μL), MeOH (250 μL), 35° C., 15 min. Radiochemical purity (RCP) was calculated by dividing the integrated area of the ¹⁸F-labeled product peak by the total integrated area of all ¹⁸F-labeled peaks, as determined by radio-HPLC. The decay-corrected radiochemical yield (RCY) was calculated by dividing final activity of the labeled product by starting [¹⁸F]1 activity, multiplied by the RCP. Identity of each labeled embodiment was confirmed by co-injection with the ¹⁹F-reference standard. ^{a}substrate (3 μmol). ^{b}substrate (0.62 μmol), Tris buffer pH 8.0 (562 μL), MeOH (188 μL). cMeCN (500 μL), H2O (500 μL).

FIG. 3. Schematic showing an embodiment of the invention where the chemical reaction uses an oxidative addition reagent comprising a compound having a general formula as shown in this figure to label a peptide.

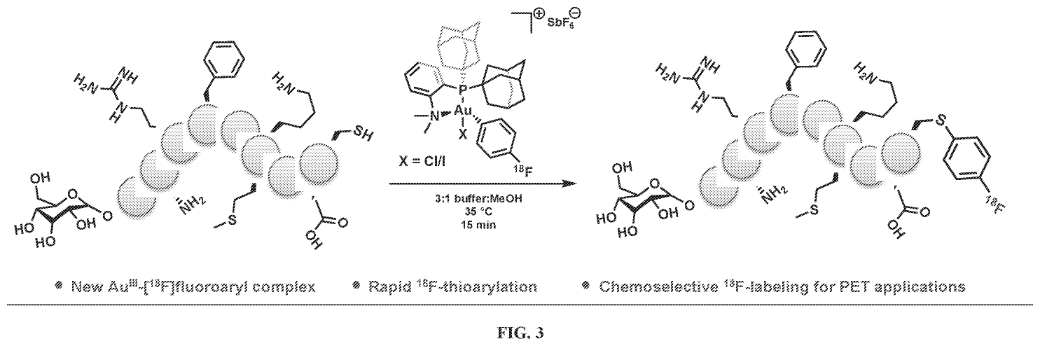

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following provides illustrative embodiments of the invention. All publications mentioned herein (e.g., McDaniel et al., Org. Lett. 2022, 24, 28, 5132-5136 Jul. 8, 2022) are incorporated herein by reference to disclose and describe aspects, methods and/or materials in connection with the cited publications.

The rapid kinetics and high chemoselectivity of transition-metal-based transformations have resulted in major advances in organic synthesis, in particular for the modification of complex small molecules.^{1, 2} In the context of ¹⁸F-labeling, significant effort has been devoted to the development of transition-metal mediated radiofluorination methods, often translated from modern fluorine-19 related approaches.³⁻⁵ Importantly, the translation of fluorine-19 to fluorine-18 chemistry presents distinct challenges that are non-trivial and rigorous optimization is generally required for smooth translation to radiochemistry.⁴ Perhaps the most notable obstacle is that ¹⁸F is always the limiting reagent and is in nanomole or lower quantities amongst a large excess of other reagents. Additionally, chemical modifications must be conducted quickly, ideally within minutes, due to the radioactive decay and half-life of ¹⁸F.

Over the last decade, reports exploiting the redox activity of transition-metals such as Pd, Ni and Cu to lower the barrier for C—¹⁸F bond formation have surged.^{3, 4, 6}-¹⁷ In particular, Cu-mediated methods have found wide use in the construction of ¹⁸F-labeled small molecules for positron emission tomography (PET) imaging applications.^{10, 18, 19} Modern Cu-mediated methods have become a truly powerful advancement in radiochemical synthesis, unlocking access to radiolabeled constructs that were previously inaccessible. However, metal-based modifications employing unprotected peptides for direct radiofluorination processes are scarce.²⁰⁻²⁷

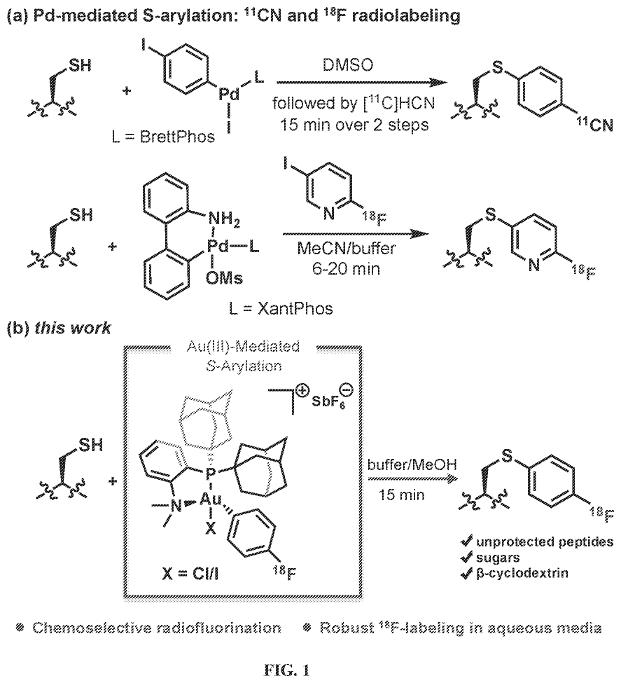

The unique properties of cysteine, largely its thiol reactivity and low natural abundance, have stimulated efforts toward the chemoselective bioconjugation of this key residue.^{28, 29} Pioneering work by the Buchwald and Pentelute groups demonstrating palladium-mediated cysteine arylation to afford S-aryl bioconjugates has encouraged the development of Pd-based strategies for labeling peptides with positron-emitting radioisotopes, such as ¹¹C or ¹⁸F.³⁰⁻³² In the context of ¹¹C-labeling, Hooker and Buchwald utilized a biarylphosphine supported Pd(II)-complex to prepare ¹¹CN-labeled unprotected peptides (FIG. 1a).³³ The Pd-mediated sequential cross-coupling proceeds with initial S-arylation of the cysteine-containing peptides followed by direct ¹¹C-cyanation. In addition, Neumaier recently reported a Pd-mediated cysteine S-arylation using the XantPhos Pd G3 system with 2-[¹⁸F]fluoro-5-iodopyridine (FIG. 1a).²⁷ The radiolabeled aryl iodide was synthesized from a DABCO precursor and obtained after solid-phase extraction (SPE) with a moderate molar activity of 29 GBq·μmol⁻¹ and could be directly used for bioconjugation, delivering a remarkably quick overall procedure. However, nonradioactive impurities formed in the initial radiofluorination step were shown to impede the consecutive S-arylation step. To sequentially perform the protocol and maintain high conversion during S-arylation, minimal DABCO precursor was used, triggering a modest RCY of 2-[¹⁸F]fluoro-5-iodopyridine.

Recently, Au(III)-aryl oxidative addition complexes supported by the aminophosphine Me-DalPhos ligand (Me-DalPhos=(Ad₂P(₀-C₆H₄)NMe₂)) provided rapid access to S-aryl bioconjugates under mild conditions at ambient temperature.³⁴⁻³⁶ The air-stable organometallic Au(III) complexes were prepared in a straightforward one-step synthesis from commercial (Me-DalPhos)AuCl with a 3-fold excess of aryl iodides, conducted at −20° C.³⁷ The extremely rapid reaction rate of S-arylation for this system (˜10⁴ M⁻¹s⁻¹) suggests this chemistry can be potentially amenable to transformations where rapid kinetics is critical. Importantly, competition experiments revealed superior kinetics for the Au-mediated system over the Pd-mediated system, with a ratio of 9:1.34 We therefore hypothesized that an ¹⁸F-labeled Au(III)-aryl oxidative addition complex could be prepared by using a radiolabeled aryl iodide such as 4-[¹⁸F]fluoroiodobenzene and subsequently used for rapid radiolabeling of biomolecules.³⁸

Despite differences in the stoichiometry by several orders of magnitude when transitioning to fluorine-18, we reasoned that the high efficiency of the oxidative addition and the rapid reaction kinetics of the Au(III) arylation could provide a powerful platform for the chemoselective radiofluorination of thiols. Here, we report the synthesis of a Au(III)-[¹⁸F]fluoroaryl complex and its application toward Au-mediated radiofluorination of thiol-containing substrates to afford stable S-[¹⁸F]fluoroaryl bioconjugates (FIG. 1b). This approach is, to our knowledge, the first gold-mediated methodology for chemoselective ¹⁸F-labeling of thiol-containing substrates.³⁹

As discussed below, the invention disclosed herein has a number of embodiments. Embodiments of the invention include, for example, compositions of matter including an oxidative addition reagent. Typically, this oxidative addition reagent comprises Au(III), a ligand bound to Au(III), the ligand comprising a phosphine, polydentate and/or monodentate ligand; and an aryl or heterocycle ring coupled to a ¹⁸F or ¹¹C moiety. In such compositions, when the oxidative addition reagent is combined with a biomolecule comprising a sulfur or selenium atom in solution; the oxidative addition reagent reacts with the biomolecule in the solution so as to couple the ¹⁸F or the ¹¹C moiety to the biomolecule. In certain embodiments of the invention, the compositions further comprise a polypeptide selected to comprise a sulfur atom; and/or a polysaccharide selected to comprise a sulfur atom. In some embodiments of the invention, the polypeptide is an unprotected polypeptide; and/or the polypeptide and/or the polysaccharide is coupled to an ¹⁸F moiety. In certain embodiments of the invention, the compositions comprise an aqueous media, an aqueous buffering agent; and/or an alcohol.

Embodiments of the invention also include methods of making the compositions disclosed herein. Typically, these methods comprise combining together to make an oxidative addition reagent, Au(III), a ligand bound to Au(III), the ligand comprising a phosphine, polydentate and/or monodentate ligand; and an aryl or heterocycle ring coupled to a ¹⁸F or ¹¹C moiety. Optionally, the compositions further comprise a polypeptide selected to comprise a sulfur atom; and/or a polysaccharide selected to comprise a sulfur atom. In certain embodiments of the invention, the method comprises including in the combination at least one of an aqueous media, an aqueous buffering agent (e.g., a PBS or TRIS buffering agent), and/or an alcohol. In illustrative working embodiments of the invention, the composition comprises a TRIS buffer/methanol solvent system.

Embodiments of the invention include methods of coupling ¹⁸F or ¹¹C to a sulfur atom. Typically these methods comprise combining together an oxidative addition reagent comprising Au(III), a ligand bound to Au(III), the ligand comprising a phosphine, polydentate and/or monodentate ligand; and an aryl or heterocycle ring coupled to a ¹⁸F or ¹¹C moiety, typically in an aqueous media. These methods can further include in the combination a polypeptide selected to comprise a sulfur atom; and/or a polysaccharide selected to comprise a sulfur atom. In such embodiments, the combination undergoes an oxidative addition reaction such that ¹⁸F is coupled to a sulfur atom present on the polypeptide selected to comprise a sulfur atom; and/or the polysaccharide selected to comprise a sulfur atom. In certain of these methods, the oxidative addition reaction is selected to occur at a temperature of 45° C. or below. In certain of these methods, the oxidative addition reaction is allowed to proceed for less than 60, 30 or 15 minutes. In some embodiments the method generates [¹⁸F] coupled polypeptides or polysaccharides in an at least 80% radiochemical yield (RCY).

Other embodiments of the invention comprising utilizing a [¹⁸F] coupled polypeptide or polysaccharide made by a method of the invention in a positron emission tomography (PET) process. Such embodiments include, for example, methods for imaging a biological target by PET scanning, the method comprising combining a¹⁸F labeled imaging agent generated by a method disclosed herein with the biological target, and using the ¹⁸F labeled imaging agent to image the target. Further aspects and embodiments of the invention are discussed below.

Our strategy in these studies first sought to prepare a radiolabeled aryl iodide that could undergo oxidative addition with the (Me-DalPhos)AuCl complex in the presence of AgSbF₆ to generate the radiolabeled Au(III)-aryl complex, [(Me-DalPhos)Au(4-└¹⁸F┘fluorobenzene)Cl┘└SbF₆┘ ([¹⁸F]1).^{34, 37} Synthesis of 4-[¹⁸F]fluoroiodobenzene ([¹⁸F]2) was achieved using a one-step radiofluorination protocol via a spirocyclic hypervalent iodonium ylide (Table 1).^{40, 41} Following a slightly modified literature protocol, iodonium ylide 3 was prepared and subsequently subjected to radiofluorination.^{42, 43} Preparation of [¹⁸F]2 was fully automated on the ELIXYS FLEX/CHEM radiochemical synthesis module (Sofie Biosciences) and conducted using [¹⁸F]Et₄NF in DMF at 120° C. for 20 min which, after HPLC purification, furnished aryl iodide [¹⁸F]2 in 26±8% isolated radiochemical yield (RCY), decay-corrected (Table 1).

We next focused on the oxidative addition reaction to yield [¹⁸F]1 (Table 1). In contrast to 4-fluoroiodobenzene, which can be employed at 3-fold excess, 4-[¹⁸F]fluoroiodobenzene is the limiting reagent that is present in nanomolar or picomolar concentration, severely altering the stoichiometry of the oxidative addition step. Formation of [¹⁸F]1 proceeded in 38%±27% RCY upon the treatment of 4-[¹⁸F]fluoroiodobenzene in CH₂Cl₂ with (Me-DalPhos)AuCl (1.5 equiv) in the presence of AgSbF₆ (1.5 equiv) heated at 55° C. in a sealed vial for 10 min (Table 1, entry 1). We initially screened the stoichiometry of (Me-DalPhos)AuCl and AgSbF₆ and observed that lowering the stoichiometry of Au(I) to 0.9 equiv afforded [¹⁸F]1 in 95%±7% RCY at 55° C. in 10 min (Table 1, entry 3). The reaction was also evaluated in DCE at elevated temperatures and [¹⁸F]1 was obtained in comparable yields albeit at slightly extended reaction times (Table 1, entries 5-7). Of note, these reactions were performed in a sealed reaction vial with no rigorous exclusion of oxygen or water and conducted using commercial, unpurified solvents. Precursor 3 showed excellent stability when stored in the dark at −20° C. for up to 18 months with no detectable degradation or loss in RCY. The Au(I) complex could be stored on the benchtop and the AgSbF₆ in the glovebox with exclusion from light for up to 3 months and used with no detectable degradation.

Product identity and purity of [¹⁸F]1 were determined by analytical HPLC analysis, comparing the radio-trace of [¹⁸F]1 with the UV-trace of the ¹⁹F-reference standard, via coinjection. Rapid and clean conversion of 4-[¹⁸F]fluoroiodobenzene to [¹⁸F]1 enabled its direct use without the need for HPLC purification. The crude reaction mixture containing [¹⁸F]1 was simply filtered and concentrated under mild heating to afford [¹⁸F]1, which was directly used in subsequent thioarylation reactions (see SI Figure S9 in McDaniel et al., Org. Lett. 2022, 24, 28, 5132-5136 Jul. 8, 2022).

The reactivity of the novel Au(III)-complex, [¹⁸F]1, was examined and optimized with L-glutathione as a model peptide substrate (Table 2). Initial thioarylation was observed in 16%±13% RCY upon treatment of L-glutathione 4 (16 μmol) with [¹⁸F]1 in PBS buffer (pH 7.4) at 23° C. in 30 min (Table 2, entry 1). A buffer screen revealed that Tris buffer (pH 8.0) increased the yield to 54±16% but the reaction remained sluggish at ambient temperature (Table 2, entry 3). Upon slight heating to 35-45° C., the [¹⁸F] fluoroaryl product [¹⁸F] 7 was generated in 93-95% RCY (Table 2, entries 4-5). Attempts to shorten the reaction time led to a reduction in yield with a significant drop for reactions under 15 min (Table 2, entries 6-8).

From our previous results with peptide conjugation chemistry,⁴⁴ cosolvents have proven valuable in improving reagent solubility; we predicted that a co-solvent could further boost the Au(III)-[¹⁸F]fluoroaryl solubility and facilitate complete reaction conversion. Employing a Tris buffer/methanol (3/1) solvent system improved the reaction conversion and provided the [¹⁸F]fluoroaryl conjugate [¹⁸F]7 in 97%±3% RCY in 15 min (Table 2, entry 9). Similarly, peptide substrates 5 and 6 also revealed a significant improvement in RCY with addition of methanol to the reaction mixture (Table 2, entries 10-11). High radiolabeling efficiency while using low mass amounts of peptide precursor is advantageous in the context of radiolabeling expensive peptides with limited availability, and allows for a simplified purification process of the ¹⁸F-labeled product. With sub-micromolar peptide loading, ¹⁸F-thioarylation was achieved in 70% RCY using 0.71 μmol 4 and in 52% RCY using 0.39 μmol 4 (Table 2, entries 12-13).

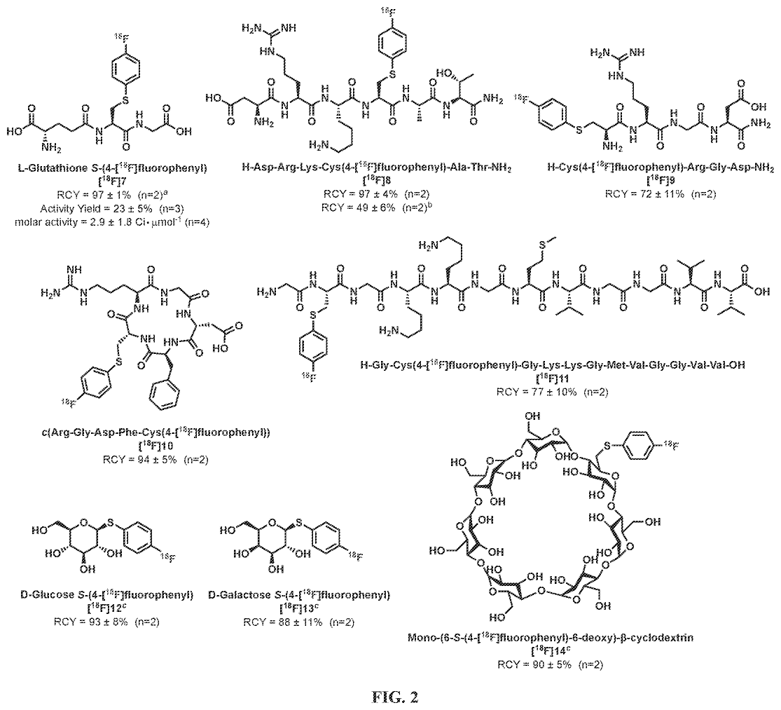

The optimized S-arylation conditions were applied to a series of thiol-containing substrates to establish the versatility and scope of our methodology (FIG. 2). High chemoselectivity for S-arylation of thiol-containing substrates in the presence of a variety of additional functional groups was observed in Tris buffer (pH 8.0)/methanol (3/1) within 15 min in 72-97% RCY. Substrates containing a free carboxylic acid, primary or secondary amine, guanidine residue, and thioether functional groups were well tolerated as well as sugar-based substrates containing free alcohols. Additionally, S-arylation of peptides in which the cysteine residue is positioned at the N-terminus ([¹⁸F]9) or within an intrachain position ([¹⁸F]10) still maintained high efficiency. Performing the ¹⁸F-thioarylation with 3 μmol L-glutathione 4, afforded the ¹⁸F-labeled conjugate [¹⁸F]7 in 97%±1% RCY (FIG. 2). A hexapeptide containing a nucleophilic lysine residue cleanly delivered the S-aryl conjugate [¹⁸F]8 in 97%±4% RCY with 7 μmol precursor loading. Notably, [¹⁸F]8 was furnished in 49%±6% RCY when using only 0.62 μmol precursor.

A critical motif utilized for noninvasive PET imaging of angiogenesis is the RGD sequence and numerous peptide-based analogues have demonstrated value, including clinical benefit.^{45, 46} The Au(III)-mediated ¹⁸F-thioarylation of peptides containing the RGD sequence was successfully executed to provide peptide conjugates [¹⁸F]9 and [¹⁸F]10 in 72%±11% and 94%±5% RCY, respectively. In addition, synthesis of an ¹⁸F-labeled β-amyloid peptide fragment⁴⁷ was successfully accomplished, using 4 μmol peptide precursor, to afford [¹⁸F]fluoroaryl conjugate [¹⁸F]11 in 77%±10% RCY. Finally, the protocol was applied to sugar-based substrates to assess compatibility with alternative thiol-containing constructs containing free alcohols. Thio-β-D-glucose and thio-β-D-galactose underwent efficient [¹⁸F]fluoroarylation in MeCN/H₂O (1/1) in 93%±8% and 88%±11% RCY, respectively.

Cyclodextrin-based polymers have been used as carrier systems for chemotherapeutics or small molecule drugs and their unique properties, such as enhanced solubility, improved pharmacokinetics and increased efficacy compared to the small molecules, have garnered interest towards utility in biomedical imaging applications.⁴⁸⁻⁵⁰ For example, a cyclodextrin polymer-based nanoparticle containing the chemotherapeutic camptothecin was labeled with ⁶⁴Cu and imaged in tumor-bearing mice to noninvasively determine multi-organ pharmacokinetics, whole-body biodistribution and tumor localization.⁵¹ Limited examples of ¹⁸F-labeled β-cyclodextrins in the literature prompted us to investigate our protocol for radiofluorination of the cyclic oligosaccharides. The Au(III)-mediated ¹⁸F-thioarylation was performed with 4 μmol of a thiolated β-cyclodextrin precursor to furnish construct [¹⁸F]14 in 90%±5% RCY.

To evaluate the practicality of our approach, S-aryl glutathione conjugate [¹⁸F]7 was synthesized using 6-8 mCi of [¹⁸F]1 and subjected to HPLC purification which afforded isolated [¹⁸F]7 in 23%±5% activity yield (non-decay-corrected, n=3) with a molar activity of 2.9±1.8 Ci·μmol⁻¹ (108±68 GBq·μmol⁻¹). ICP-OES analysis revealed that the purified product contained 2.7 ppm of Au, which is below the acceptable limit for in-human injection.⁵² The focus of this work is the design, optimization and construction of a novel Au^{III}-[¹⁸F]fluoroaryl complex for the ¹⁸F-labeling of unprotected peptides and sugars. Future work is directly aimed at automating the full protocol and conducting PET imaging studies with a labeled peptide in preclinical mouse models.

In summary, we report a robust Au(III)-└¹⁸F┘fluoroaryl reagent [¹⁸F]1 for the ¹⁸F-labeling of thiol-containing substrates via S-arylation in aqueous media. To our knowledge, this is the first Au-mediated ¹⁸F-labeling methodology of unprotected peptides and thiol-containing constructs. The practical advantages of our method are highlighted by the mild reaction conditions, broad substrate scope and rapid reaction kinetics. The oxidative addition complex [¹⁸F]1 was rapidly generated in 10 min and directly used to furnish ¹⁸F-labeled conjugates in excellent chemoselectivity and high molar activity in 15 min. The protocol was applied to a diverse range of thiol-containing substrates, including unprotected peptides, and could achieve good RCYs using sub micromolar peptide loading. This work expands on the growing space of organometallic reagents that are applied towards radiochemical modifications which demand rapid reaction rates. We anticipate the availability of [¹⁸F]1 will further advance the accessible radiolabeling space for biomedical imaging applications.

TABLE 1
Preparation of Au^{III}-[¹⁸F]Fluoroaryl Complex [¹⁸F]1

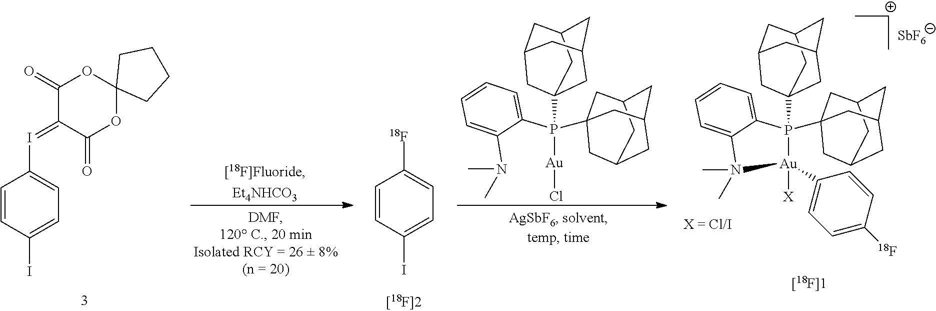

| | Au(I)/AgSbF₆ | | time | temp | RCY |
|---|---|---|---|---|---|
| entryª | (mg) | solvent | (min) | (° C.) | (%)^{c} |
| 1 | 10/5 (1.5 equiv) | DCM | 10 | 55 | 38 ± 27 |
| 2 | 8/4 (1.2 equiv) | DCM | 10 | 55 | 49 ± 15 |
| 3 | 6/3 (0.9 equiv) | DCM | 10 | 55 | 95 ± 7 |
| 4 | 6/3 (0.9 equiv) | DCM | 20 | 55 | 87 ± 16 |
| 5 | 6/3 (0.9 equiv) | DCE | 10 | 60 | 87 ± 8 |
| 6 | 6/3 (0.9 cquiv) | DCE | 10 | 80 | 83 ± 15 |
| 7 | 6/3 (0.9 equiv) | DCE | 20 | 80 | 94 ± 6 |

^{a}Conditions: [18F]2 (~500 μCi) per reaction, solvent (1.5 mL).
^{b}equiv are relative to ylide precursor 3.
^{c}RCY was determined by radio-TLC analysis of complex [18F]1, n > 3 for all entries.

TABLE 2
Thio Arylation of L-Glutathione with Au^{III}-[¹⁸F]Fluoroaryl Complex [¹⁸F]1

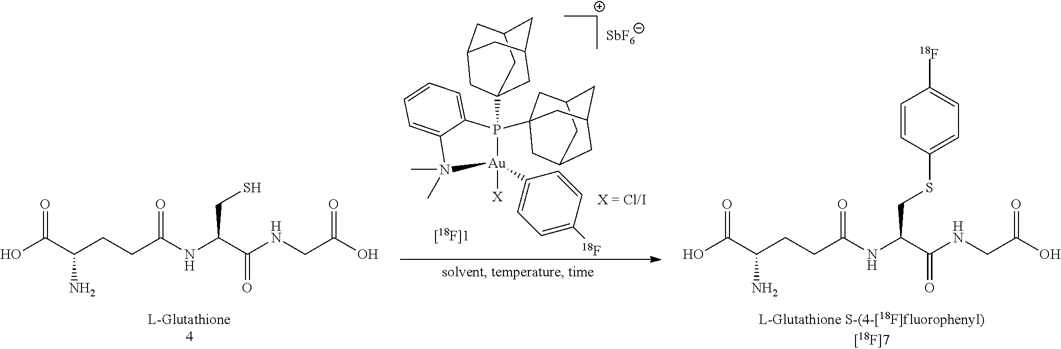

| | | time | temp. | RCY |
|---|---|---|---|---|
| entry^{a} | solvent | (min) | (° C.) | (%)^{b} |
| 1 | PBS pH 7.4 | 30 | 23 | 16 ± 13 |
| 2 | HEPES pH 7.3 | 30 | 23 | 43 ± 18 |
| 3 | Tris pH 8.0 | 30 | 23 | 54 ± 16 |
| 4 | Tris pH 8.0 | 30 | 35 | 93 ± 1 |
| 5 | Tris pH 8.0 | 30 | 45 | 95 ± 1 |
| 6 | Tris pH 8.0 | 20 | 35 | 72 ± 14 |
| 7 | Tris pH 8.0 | 15 | 35 | 78 ± 12 |
| 8 | Tris pH 8.0 | 10 | 35 | 44 ± 1 |
| 9 | Tris pH 8.0/MeOH, 3/1 | 15 | 35 | 97 ± 3 |
| 10^{c} | Tris pH 8.0/MeOH, 3/1 | 15 | 35 | 97 ± 4 |
| 11^{d} | Tris pH 8.0/MeOH, 3/1 | 15 | 35 | 91 ± 5 |
| 12^{e} | Tris pH 8.0/MeOH, 3/1 | 15 | 35 | 70 |
| 13^{f} | Tris pH 8.0/MeOH, 3/1 | 15 | 35 | 52 |

ªConditions: Au^{III} complex [¹⁸F]1 (~1 mCi) per reaction, L-glutathione 4 (16 μmol), solvent (1 mL).
^{b}non-isolated RCY is estimated by radio-HPLC analysis of crude peptide [¹⁸F]7, n = 2-6.
^{c}peptide = H—Asp—Arg—Lys—Cys—Ala—Thr—NH₂ 5 (7 μmol).
^{d}peptide = H—Cys—Arg—Gly—Asp—NH₂ 6 (11 μmol).
^{e}L-glutathione 4 (0.71 μmol), n = 1.
^{f}L-glutathione 4 (0.39 μmol), n = 1.

### REFERENCES

(1) Crabtree, R. H., In The Organometallic Chemistry of the Transition Metals, 6th ed.; Wiley, 2014; pp i-xvi.
(2) Diederich, F.; Stang, P. J., Metal-catalyzed cross-coupling reactions; Wiley & Sons, 2008.
(3) Halder, R.; Ritter, T. ¹⁸F-Fluorination: Challenge and Opportunity for Organic Chemists. J. Org. Chem. 2021, 86 (20), 13873-13884.
(4) Brooks, A. F.; Topczewski, J. J.; Ichiishi, N.; Sanford, M. S.; Scott, P. J. H. Late-stage [¹⁸F] fluorination: new solutions to old problems. Chem. Sci. 2014, 5 (12), 4545-4553.
(5) Sanford, M. S.; Scott, P. J. H. Moving Metal-Mediated ¹⁸F-Fluorination from Concept to Clinic. ACS Cent. Sci. 2016, 2 (3), 128-130.
(6) Lee, E.; Kamlet, A. S.; Powers, D. C.; Neumann, C. N.; Boursalian, G. B.; Furuya, T.; Choi, D. C.; Hooker, J. M.; Ritter, T. A Fluoride-Derived Electrophilic Late-Stage Fluorination Reagent for PET Imaging. Science 2011, 334 (6056), 639-642.
(7) Lee, E.; Hooker, J. M.; Ritter, T. Nickel-Mediated Oxidative Fluorination for PET with Aqueous [¹⁸F] Fluoride. J. Am. Chem. Soc. 2012, 134 (42), 17456-17458.
(8) Tredwell, M.; Preshlock, S. M.; Taylor, N. J.; Gruber, S.; Huiban, M.; Passchier, J.; Mercier, J.; Génicot, C.; Gouverneur, V. A General Copper-Mediated Nucleophilic ¹⁸F Fluorination of Arenes. Angew. Chem. Int. Ed. 2014, 53 (30), 7751-7755.
(9) Ichiishi, N.; Brooks, A. F.; Topczewski, J. J.; Rodnick, M. E.; Sanford, M. S.; Scott, P. J. H. Copper-Catalyzed [¹⁸F] Fluorination of (Mesityl)(aryl)iodonium Salts. Org. Lett. 2014, 16 (12), 3224-3227.
(10) Preshlock, S.; Calderwood, S.; Verhoog, S.; Tredwell, M.; Huiban, M.; Hienzsch, A.; Gruber, S.; Wilson, T. C.; Taylor, N. J.; Cailly, T.; Schedler, M.; Collier, T. L.; Passchier, J.; Smits, R.; Mollitor, J.; Hoepping, A.; Mueller, M.; Genicot, C.; Mercier, J.; Gouverneur, V. Enhanced copper-mediated ¹⁸F-fluorination of aryl boronic esters provides eight radiotracers for PET applications. Chem. Commun. 2016, 52 (54), 8361-8364.
(11) Mossine, A. V.; Brooks, A. F.; Makaravage, K. J.; Miller, J. M.; Ichiishi, N.; Sanford, M. S.; Scott, P. J. H. Synthesis of [¹⁸F] Arenes via the Copper-Mediated [¹⁸F]Fluorination of Boronic Acids. Org. Lett. 2015, 17 (23), 5780-5783.
(12) Neumann, C. N.; Hooker, J. M.; Ritter, T. Concerted nucleophilic aromatic substitution with ¹⁹F- and ¹⁸F-. Nature 2016, 534 (7607), 369-373.
(13) Beyzavi, M. H.; Mandal, D.; Strebl, M. G.; Neumann, C. N.; D'Amato, E. M.; Chen, J.; Hooker, J. M.; Ritter, T. ¹⁸F-Deoxyfluorination of Phenols via Ru π-Complexes. ACS Cent. Sci. 2017, 3 (9), 944-948.
(14) McCammant, M. S.; Thompson, S.; Brooks, A. F.; Krska, S. W.; Scott, P. J. H.; Sanford, M. S. Cu-Mediated C—H ¹⁸F-Fluorination of Electron-Rich (Hetero)arenes. Org. Lett. 2017, 19 (14), 3939-3942.
(15) Huang, X.; Liu, W.; Ren, H.; Neelamegam, R.; Hooker, J. M.; Groves, J. T. Late Stage Benzylic C—H Fluorination with [¹⁸F]Fluoride for PET Imaging. J. Am. Chem. Soc. 2014, 136 (19), 6842-6845.
(16) Makaravage, K. J.; Brooks, A. F.; Mossine, A. V.; Sanford, M. S.; Scott, P. J. H. Copper-Mediated Radiofluorination of Arylstannanes with [¹⁸F]KF. Org. Lett. 2016, 18 (20), 5440-5443.
(17) Zhang, M.; Li, S.; Zhang, H.; Xu, H. Research progress of ¹⁸F labeled small molecule positron emission tomography (PET) imaging agents. Eur. J. Med. Chem. 2020, 205, 112629.
(18) Mossine, A. V.; Tanzey, S. S.; Brooks, A. F.; Makaravage, K. J.; Ichiishi, N.; Miller, J. M.; Henderson, B. D.; Skaddan, M. B.; Sanford, M. S.; Scott, P. J. H. One-pot synthesis of high molar activity 6-[¹⁸F]fluoro-l-DOPA by Cu-mediated fluorination of a BPin precursor. Org. Biomol. Chem. 2019, 17 (38), 8701-8705.
(19) Wright, J. S.; Kaur, T.; Preshlock, S.; Tanzey, S. S.; Winton, W. P.; Sharninghausen, L. S.; Wiesner, N.; Brooks, A. F.; Sanford, M. S.; Scott, P. J. H. Copper-mediated late-stage radiofluorination: five years of impact on preclinical and clinical PET imaging. Clin. Transl. Imaging 2020, 8 (3), 167-206.
(20) Gao, Z.; Gouverneur, V.; Davis, B. G. Enhanced Aqueous Suzuki-Miyaura Coupling Allows Site-Specific Polypeptide ¹⁸F-Labeling. J. Am. Chem. Soc. 2013, 135 (37), 13612-13615.
(21) Way, J. D.; Bergman, C.; Wuest, F. Sonogashira cross-coupling reaction with 4-[¹⁸F]fluoroiodobenzene for rapid ¹⁸F-labelling of peptides. Chem. Commun. 2015, 51 (18), 3838-3841.
(22) Gray, E. E.; Nielsen, M. K.; Choquette, K. A.; Kalow, J. A.; Graham, T. J. A.; Doyle, A. G. Nucleophilic (Radio)Fluorination of α-Diazocarbonyl Compounds Enabled by Copper-Catalyzed H—F Insertion. J. Am. Chem. Soc. 2016, 138 (34), 10802-10805.
(23) Yuan, Z.; Nodwell, M. B.; Yang, H.; Malik, N.; Merkens, H.; Bénard, F.; Martin, R. E.; Schaffer, P.; Britton, R. Site-Selective, Late-Stage C—H ¹⁸F-Fluorination on Unprotected Peptides for Positron Emission Tomography Imaging. Angew. Chem. Int. Ed. 2018, 57 (39), 12733-12736.
(24) Verhoog, S.; Kee, C. W.; Wang, Y.; Khotavivattana, T.; Wilson, T. C.; Kersemans, V.; Smart, S.; Tredwell, M.; Davis, B. G.; Gouverneur, V. ¹⁸F-Trifluoromethylation of Unmodified Peptides with 5-¹⁸F-(Trifluoromethyl)dibenzothiophenium Trifluoromethanesulfonate. J. Am. Chem. Soc. 2018, 140 (5), 1572-1575.
(25) Rickmeier, J.; Ritter, T. Site-Specific Deoxyfluorination of Small Peptides with [¹⁸F]Fluoride. Angew. Chem. Int. Ed. 2018, 57 (43), 14207-14211.
(26) Kee, C. W.; Tack, O.; Guibbal, F.; Wilson, T. C.; Isenegger, P. G.; Imiolek, M.; Verhoog, S.; Tilby, M.; Boscutti, G.; Ashworth, S.; Chupin, J.; Kashani, R.; Poh, A. W. J.; Sosabowski, J. K.; Macholl, S.; Plisson, C.; Cornelissen, B.; Willis, M. C.; Passchier, J.; Davis, B. G.; Gouverneur, V. ¹⁸F-Trifluoromethanesulfinate Enables Direct C—H ¹⁸F-Trifluoromethylation of Native Aromatic Residues in Peptides. J. Am. Chem. Soc. 2020, 142 (3), 1180-1185.
(27) Humpert, S.; Omrane, M. A.; Urusova, E. A.; Gremer, L.; Willbold, D.; Endepols, H.; Krasikova, R. N.; Neumaier, B.; Zlatopolskiy, B. D. Rapid ¹⁸F-labeling via Pd-catalyzed S-arylation in aqueous medium. Chem. Commun. 2021, 57 (29), 3547-3550.
(28) Koniev, O.; Wagner, A. Developments and recent advancements in the field of endogenous amino acid selective bond forming reactions for bioconjugation. Chem. Soc. Rev. 2015, 44 (15), 5495-5551.
(29) Krishnan, H. S.; Ma, L.; Vasdev, N.; Liang, S. H. ¹⁸F-Labeling of Sensitive Biomolecules for Positron Emission Tomography. Chem. Eur. J. 2017, 23 (62), 15553-15577.
(30) Vinogradova, E. V.; Zhang, C.; Spokoyny, A. M.; Pentelute, B. L.; Buchwald, S. L. Organometallic palladium reagents for cysteine bioconjugation. Nature 2015, 526 (7575), 687-691.
(31) Rojas, A. J.; Pentelute, B. L.; Buchwald, S. L. Water-Soluble Palladium Reagents for Cysteine S-Arylation under Ambient Aqueous Conditions. Org. Lett. 2017, 19 (16), 4263-4266.
(32) Rojas, A. J.; Zhang, C.; Vinogradova, E. V.; Buchwald, N. H.; Reilly, J.; Pentelute, B. L.; Buchwald, S. L. Divergent unprotected peptide macrocyclisation by palladium-mediated cysteine arylation. Chem. Sci. 2017, 8 (6), 4257-4263.
(33) Zhao, W.; Lee, H. G.; Buchwald, S. L.; Hooker, J. M. Direct ¹¹CN-Labeling of Unprotected Peptides via Palladium-Mediated Sequential Cross-Coupling Reactions. J. Am. Chem. Soc. 2017, 139 (21), 7152-7155.
(34) Messina, M. S.; Stauber, J. M.; Waddington, M. A.; Rheingold, A. L.; Maynard, H. D.; Spokoyny, A. M. Organometallic Gold (III) Reagents for Cysteine Arylation. J. Am. Chem. Soc. 2018, 140 (23), 7065-7069.
(35) Stauber, J. M.; Qian, E. A.; Han, Y.; Rheingold, A. L.; Král, P.; Fujita, D.; Spokoyny, A. M. An Organometallic Strategy for Assembling Atomically Precise Hybrid Nanomaterials. J. Am. Chem. Soc. 2020, 142 (1), 327-334.
(36) Stauber, J. M.; Rheingold, A. L.; Spokoyny, A. M. Gold(III) Aryl Complexes as Reagents for Constructing Hybrid Peptide-Based Assemblies via Cysteine S-Arylation. Inorg. Chem. 2021, 60 (7), 5054-5062.
(37) Zeineddine, A.; Estévez, L.; Mallet-Ladeira, S.; Miqueu, K.; Amgoune, A.; Bourissou, D. Rational development of catalytic Au(I)/Au(III) arylation involving mild oxidative addition of aryl halides. Nat. Commun. 2017, 8 (1), 565.
(38) Way, J. D.; Wuest, F. Automated radiosynthesis of no-carrier-added 4-[¹⁸F]fluoroiodobenzene: a versatile building block in ¹⁸F radiochemistry. J. Labelled Comp. Radiopharm. 2014, 57 (2), 104-109.
(39) Huang, B.; Hu, M.; Toste, F. D. Homogeneous Gold Redox Chemistry: Organometallics, Catalysis, and Beyond. Trends Chem. 2020, 2 (8), 707-720.
(40) Rotstein, B. H.; Stephenson, N. A.; Vasdev, N.; Liang, S. H. Spirocyclic hypervalent iodine (III)-mediated radiofluorination of non-activated and hindered aromatics. Nat. Commun. 2014, 5, 4365.
(41) Rotstein, B. H.; Wang, L.; Liu, R. Y.; Patteson, J.; Kwan, E. E.; Vasdev, N.; Liang, S. H. Mechanistic Studies and Radiofluorination of Structurally Diverse Pharmaceuticals with Spirocyclic Iodonium(III) Ylides. Chem. Sci. 2016, 7 (7), 4407-4417.
(42) Koser, G. F.; Wettach, R. H. [Hydroxy(tosyloxy)iodo]benzene, a versatile reagent for the mild oxidation of aryl iodides at the iodine atom by ligand transfer. J. Org. Chem. 1980, 45 (8), 1542-1543.
(43) Jiang, J.; Zeng, W. Synthesis and Crystal Structures of Two New Oxaspirocyclic Compounds. Crystals 2016, 6 (10), 134.
(44) Ma, G.; McDaniel, J. W.; Murphy, J. M. One-Step Synthesis of [¹⁸F]Fluoro-4-(vinylsulfonyl)benzene: A Thiol Reactive Synthon for Selective Radiofluorination of Peptides. Org. Lett. 2021, 23 (2), 530-534.
(45) Chen, H.; Niu, G.; Wu, H.; Chen, X. Clinical Application of Radiolabeled RGD Peptides for PET Imaging of Integrin αvβ3. Theranostics 2016, 6, 78-92.
(46) Chin, F. T.; Shen, B.; Liu, S.; Berganos, R. A.; Chang, E.; Mittra, E.; Chen, X.; Gambhir, S. S. First Experience with Clinical-Grade [¹⁸F]FPP(RGD)₂: An Automated Multi-step Radiosynthesis for Clinical PET Studies. Mol. Imaging Biol. 2012, 14 (1), 88-95.
(47) Vandenberghe, R.; Adamczuk, K.; Dupont, P.; Laere, K. V.; Chételat, G. Amyloid PET in clinical practice: Its place in the multidimensional space of Alzheimer's disease. Neuroimage Clin. 2013, 2, 497-511.
(48) Lai, W.-F.; Rogach, A. L.; Wong, W.-T. Chemistry and engineering of cyclodextrins for molecular imaging. Chem. Soc. Rev. 2017, 46 (20), 6379-6419.
(49) Shepelytskyi, Y.; Newman, C. J.; Grynko, V.; Seveney, L. E.; DeBoef, B.; Hane, F. T.; Albert, M. S. Cyclodextrin-Based Contrast Agents for Medical Imaging. Molecules 2020, 25 (23), 5576.
(50) Bartlett, D. W.; Su, H.; Hildebrandt, I. J.; Weber, W. A.; Davis, M. E. Impact of tumor-specific targeting on the biodistribution and efficacy of siRNA nanoparticles measured by multimodality in vivo imaging. Proc. Natl. Acad. Sci. U.S.A. 2007, 104 (39), 15549-15554.
(51) Schluep, T.; Hwang, J.; Hildebrandt, I. J.; Czernin, J.; Choi, C. H. J.; Alabi, C. A.; Mack, B. C.; Davis, M. E. Pharmacokinetics and tumor dynamics of the nanoparticle IT-101 from PET imaging and tumor histological measurements. Proc. Natl. Acad. Sci. U.S.A. 2009, 106 (27), 11394-11399.
(52) For guidelines of residual metal contents, see: http://www.ema.europa.eu/docs/en_GB/document_library/Scientific_guideline/2009/0 9/WC500003587.pdf

1. A composition of matter including an oxidative addition reagent comprising:
(a) Au(III);
(b) a ligand bound to Au(III), the ligand comprising a phosphine, polydentate and/or monodentate ligand; and
(c) an aryl or heterocycle ring coupled to a ¹⁸F or ¹¹C moiety;
wherein, when combined with a biomolecule comprising a sulfur or selenium atom in solution; the oxidative addition reagent reacts with the biomolecule in the solution so as to couple the ¹⁸F or the ¹¹C moiety to the biomolecule.

2. The composition of claim 1, further comprising:
a polypeptide selected to comprise a sulfur atom; and/or
a polysaccharide selected to comprise a sulfur atom.

3. The composition of claim 2, further comprising:
an aqueous media;
an aqueous buffering agent; and/or
an alcohol.

4. The composition of claim 2, wherein:
the polypeptide is an unprotected polypeptide; and/or
the polypeptide and/or the polysaccharide is coupled to an ¹⁸F moiety.

5. The composition of claim 1, wherein the oxidative addition reagent comprises a compound having a general formula:

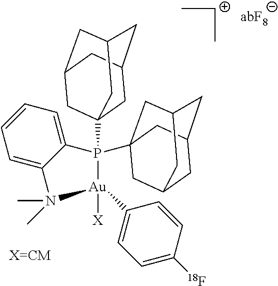

6. A method of making a composition, comprising combining together:
an oxidative addition reagent comprising:
(a) Au(III);
(b) a ligand bound to Au(III), the ligand comprising a phosphine, polydentate and/or monodentate ligand; and
(c) an aryl or heterocycle ring coupled to a ¹⁸F or ¹¹C moiety;
a polypeptide selected to comprise a sulfur atom; and/or
a polysaccharide selected to comprise a sulfur atom; such that the composition is made.

7. The method of claim 6, wherein the method comprises including in the combination at least one of:
an aqueous media;
an aqueous buffering agent; and/or
an alcohol.

8. The method of claim 7, wherein the composition comprises a PBS or TRIS buffering agent.

9. The method of claim 8, wherein the composition comprises a TRIS buffer/methanol solvent system.

10. A method of coupling ¹⁸F or ¹¹C to a sulfur atom, the method comprising combining together:
an oxidative addition reagent comprising:
(a) Au(III);
(b) a ligand bound to Au(III), the ligand comprising a phosphine, polydentate and/or monodentate ligand; and
(c) an aryl or heterocycle ring coupled to a ¹⁸F or ¹¹C moiety;
an aqueous media;
a polypeptide selected to comprise a sulfur atom; and/or
a polysaccharide selected to comprise a sulfur atom; wherein:
the combination undergoes an oxidative addition reaction such that ¹⁸F is coupled to a sulfur atom present on the polypeptide selected to comprise a sulfur atom; and/or the polysaccharide selected to comprise a sulfur atom.

11. The method of claim 10, wherein:
the oxidative addition reaction occurs at a temperature of 45° C. or below; and/or
the oxidative addition reaction is allowed to proceed for less than 60, 30 or 15 minutes.

12. The method of claim 11, wherein the method generates [¹⁸F] coupled polypeptides or polysaccharides in an at least 80% radiochemical yield (RCY).

13. The method of claim 11, wherein the oxidative addition reagent comprises a ¹⁸F labelled aryl iodide,

14. The method of claim 10, further comprising utilizing the [¹⁸F] coupled polypeptides or polysaccharides in a positron emission tomography (PET) process.

15. A method for imaging a biological target by PET scanning, the method comprising combining a¹⁸F labeled imaging agent generated by the method of claim 6 with the biological target, and using the ¹⁸F labeled imaging agent to image the target.
